(19) United States
(12) Patent Application Publication
(10) Pub. No.: US 2017/0209299 A1
(43) Pub. Date: Jul. 27, 2017

(54) Glove for Preventing Sucking of Thumb and Fingers

(71) Applicants: Kathleen Szopa, Clovis, CA (US); Paul A. Szopa, Clovis, CA (US)

(72) Inventors: Kathleen Szopa, Clovis, CA (US); Paul A. Szopa, Clovis, CA (US)

(21) Appl. No.: 13/208,739

(22) Filed: Aug. 12, 2011

(51) Int. Cl. A61F 5/50; A41D 19/00

(52) U.S. Cl. CPC A61F 5/50; A41D 19/0013; A41D 19/0048; A41D 2400/22

(57) ABSTRACT

A glove for preventing the wearer from sucking a thumb or one or more fingers comprises a glove body made out of fabric that has a hand portion which receives the hand and a digit portion which receives the thumb and fingers. The digit portion comprises a tubular, open ended digit receiving member for each digit. Some of these are configured as affected members for receiving the affected digits, those being the digits the wearer normally sucks. The affected members extend substantially the entire length of their respective digit. At least one anti-sucking component, preferably an elongated tube, extends along the length of the affected member, but not completely to its open end. The channel defined by the anti-sucking component prevents the wearer from achieving a vacuum against the affected member, thereby preventing suction. The glove has a securing mechanism that prevents the wearer from easily removing the glove.

### CROSS-REFERENCE TO RELATED APPLICATIONS

None.

### STATEMENT REGARDING FEDERALLY SPONSORED RESEARCH

Not Applicable.

### BACKGROUND OF THE INVENTION

A. Field of the Invention

The field of the present invention relates generally to devices for preventing thumb and finger sucking. In particular, the present invention relates to such devices that prevent the person from obtaining a vacuum at his or her thumb or finger to discourage any sucking activity and assist the person break the habit of thumb or finger sucking. More particularly, the present invention relates to such devices that are configured in the form of a glove that is configured to allow the person to fully utilize his or her hand.

B. Background

As well known to many parents, the sucking of the thumb or one or more of the fingers is a relatively common habit among infants and young children, particularly those children who are less than approximately two years old. Although most children outgrow the habit of sucking their thumb or fingers while they are relatively young, some children continue to suck their thumb and/or one or more of their fingers until well into their early school years. Unfortunately, thumb or finger sucking can result in damage to the child's teeth and jawbone if they continue such activity for too long of a period. For instance, if the child continues to suck their thumb and/or fingers after the age of three, which is when the permanent teeth typically start to come in, the sucking action can cause their teeth to grow out of alignment and position, can reshape the jawbone due to it being relatively soft and pliable, can affect the growth of the roof the child's mouth (his or her palate) and lead to problems with regard to chewing, speaking and snoring. As well known, once any activity becomes a habit and remains a habit for a period of time, it becomes much harder to break the habit. As a result, many people recommend that the parent take action to discourage thumb and finger sucking while the child is still very young, such as around the time the child turns two, to prevent the activity from becoming a habit.

One of the traditional methods of discouraging thumb or finger sucking is to provide the child with a pacifier or other sucking device to replace the thumb or finger sucking. Unfortunately, this may only result in the child developing a habit for the pacifier or other device, which can result in many of the same problems as thumb or finger sucking. In addition, once the pacifier or other device is taken away, the child may simply revert to the thumb or finger sucking as a replacement. Another traditional method of discouraging thumb or finger sucking is to place a substance on the child's favorite digit to suck, typically either the thumb or a pair of fingers, such that when the child puts the thumb or fingers into his or her mouth he or she will find the taste unpleasant or even revolting and not want to suck the digit. Substances utilized for such purposes tend to be very sour, such as lemon juice or the like, tart or otherwise something the child would not want to taste. Unfortunately, this method is seriously limited in its ability to be effective because the substance can be wiped off and/or the child can suffer through the initial displeasant taste until the substance is substantially removed from the digit. In addition, many substances that are useful for this purpose can be harmful to the eyes if the child rubs the eyes with the substance on his or her hand. Some substances, such as cayenne pepper laced polish, can be harmful to some children, particularly infants. Another one of the traditional methods of discouraging thumb or finger sucking is to place a larger sized long sleeve shirt on the child and pull the sleeves over his or her hands and then pin or otherwise secure the sleeves in a closed position around the hands or place gloves on the child's hands. Unfortunately, both of these methods can usually be undone or removed by the child who is not interested in having to cease sucking his or her thumb or finger and both methods may interfere with a young child's dexterity development.

A number of prior art patents have attempted to address potential solutions to the problem of how to discourage thumb and finger sucking. Some of these devices are configured to be worn around the hand and have an extending portion that encloses or partially encloses the thumb and one or more fingers. For instance, U.S. Pat. No. 5,515,870 to Zilber discloses a thumb and finger sucking prevention device that utilizes a vacuum-breaking cylinder placed over the thumb or one or more fingers that prevents the formation of a sucking vacuum by utilizing air passages between in the cylinder or between the cylinder and the digit. The cylinder is sufficiently rigid to prevent collapsing, which would allow sucking, and is secured to the hand by means of a wristband and a strap that connects the cylinder to the wristband. Various other patents, such as U.S. Pat. No. 1,990,384 to Klohs, U.S. Pat. No. 5,474,093 to Pettiet, U.S. Pat. No. 4,396,014 to Pace, et al., U.S. Pat. No. 1,633,037 to Rood and U.S. Pat. No. 1,794,515 to Davis also describe thumb sucking prevention devices that have a generally rigid, tubular member which encloses the thumb or a finger and have one or more straps or ties that secure the device to the user's hand to prevent the user from sucking the thumb or finger. Most of these types of devices have rigid cylinder members that can inhibit the child's dexterity development, provide a chewable surface for the child to chew on, lead to chaffing of the enclosed digit and allow moisture to accumulate between the cylinder and digit.

Other prior art patents disclose glove or glove-like devices that are configured for preventing a child from sucking his or her thumb and/or one or more fingers. For instance, U.S. Pat. No. 2,783,759 to Hill discloses a glove for preventing thumb sucking that comprises a glove having open ended thumb and finger extensions with flexible strips embedded in the woven material of the extensions to prevent the lips of the infant from forming a complete vacuum around the thumb or fingers. The glove is secured to the hand with a chain and lock combination. The flexible strips appear to be such that a child could pinch them off with his or her teeth to obtain sufficient vacuum to enjoy sucking and the chain could hurt the child's teeth if he or she bit it. U.S. Patent Publication No. 2007/0028341 to Smith discloses a glove-like device for preventing thumb sucking that comprises a glove with an opening for all of the fingers to extend through to allow uncovered use of the fingers and a thumb enclosure that completely covers the thumb to prevent sucking thereof. A tie mechanism is used to secure the glove to the hand. U.S. Pat. No. 5,797,405 to Brock discloses a thumb sucking deterrent device that also comprises a glove made out of cloth with an opening for all of the fingers to extend through and thumb shield structure made out of terry cloth having outward extending loops with a chamber that receives the thumb. An anti-sucking latex rubber coating coats the thumb portion to prevent sucking. Snaps are utilized to secure the glove to the hand. U.S. Pat. No. 2,498,122 to Haniuk discloses digit sucking deterrent device comprising a mitt worn over the hand having open ended thumb and finger extensions through which the thumb and fingers extend and one or more tubular shaped guards, preferably made out of plastic, that are placed on the extending thumb/finger to prevent suction thereof. U.S. Pat. No. 5,010,901 to Pales discloses a device for preventing thumb or finger sucking that is configured as a partial glove having a tubular closed-ended digit receiving part which receives and encloses the thumb or one or more fingers and an external flange near the end of the digit receiving part to prevent insertion of the part into the mouth. The wrist portion of the glove tightly encircles the wrist to prevent its removal. Several of the above-described devices do not prevent sucking of the fingers and have a securing mechanism, such as ties or snaps, that can be easily undone by most toddlers, which would likely prevent their effective use for toddlers and older children. Other devices have removable suction prevention components that appear to be easily removed by toddlers and older children. Other devices are configured in a manner that either limits dexterity of the thumb or fingers or can be relatively easy to manipulate the hand out of the glove and, as such, will only work on children who want to stop sucking their thumb or fingers.

Although the prior art generally describes a number of different types of devices for preventing the wearer from sucking his or her thumb and/or one or more fingers, there are certain limitations to such devices that have limited their full acceptance and use. What is needed, therefore, is an improved anti-suction device that prevents the wearer from effectively sucking his or her thumb and/or one or more fingers so as to discourage such sucking activity and either break a pre-developed sucking habit or prevent the sucking from becoming a habit. The preferred device should be configured to prevent the wearer from being able to obtain a vacuum around the thumb or fingers so as to discourage him or her from wanting to suck the thumb or fingers. Preferably, the device should be configured such that the wearer, even older children, cannot easily remove the device from his or her hand or remove the anti-suction component from the device to defeat the purpose of the device. The preferred device should be configured so as to not interfere with the dexterity development of the wearer by allowing the wearer to accomplish the same movement and tasks that he or she could without the device. Preferably, the device should be adaptable for use to prevent sucking of the thumb and/or one or more of the fingers and be adaptable to a wide range of sizes and shapes of hands to accommodate different ages and sizes of children or other thumb/finger suckers.

### SUMMARY OF THE INVENTION

The glove for preventing sucking of thumb and fingers of the present invention solves the problems and provides the benefits identified above. That is to say, the present invention discloses an anti-sucking device that is configured as a glove which is worn on the user's hand with a hand portion secured to the user's wrist and a digit portion with an affected member that covers the affected digit or digits having one or more tubes disposed along the length of the affected portion to prevent the wearer from being able to create a vacuum to discourage him or her from sucking the digit or digits and prevent or break a sucking habit. The preferred embodiment also includes a securing mechanism at the wrist area of the hand portion that prevents the wearer from easily removing the glove and, thereby defeating the anti-suction benefits of the present invention. The present invention does not include any anti-suction components that can be removed by the wearer. Because the glove of the present invention allows the wearer to perform the same tasks as he or she would be able to without the glove, it will not interfere with the wearer's dexterity development. The glove of the present invention can be manufactured in a wide variety of different sizes, such as a small size for infants and toddlers and a larger size for young children and adults who may have a thumb/finger sucking problem, so the glove can comfortably fit wearer's having different sizes and shapes of hands.

In a primary embodiment of the present invention, the glove for preventing sucking of thumb and fingers comprises a glove body, which generally conforms to the shape of the wearer's hand, having a hand portion with a hand opening for receiving the wearer's hand therein and a digit portion with a plurality of open ended digit receiving members configured to receive the wearer's thumb and fingers therein. The digit receiving members are open to allow at least the tips of the wearer's fingers and thumb to extend beyond the end of the digit receiving members so he or she may use the ends of the digits for grabbing and manipulating objects. To prevent sucking of the affected digits, that being those digits (thumb or fingers) which the wearer normally sucks on, the corresponding digit receiving members, designated as affected members, extend substantially the full length of the affected digits. Those digit receiving members which receive digits that are not typically sucked on are preferably configured to not extend beyond the base of their digit. Each of the affected members have one or more anti-suction components disposed along a length of the affected member that are configured to define an air flow channel in the affected member that prevents the wearer from obtaining a vacuum when he or she sucks on the affected member or digit. In one embodiment where the thumb is the affected digit, the affected member that extends along the thumb can have two anti-suction components that are spaced apart to prevent the wearer from pinching both of the components at the same time with his or her teeth, which would block the air flow channel and allow a vacuum to be formed. The anti-suction components are positioned in the affected members such that the first end of the component does not extend completely to the open end of the affected member in order to prevent clogging or other blocking of the air flow channel defined by the anti-suction component. In one embodiment, the second end of the anti-suction components extends into the hand portion of the glove body and air is drawn therefrom. In the preferred embodiment, the anti-suction component is a tube that has the air flow channel disposed inside the tube. Preferably, any such tube is made out of materials and configured so as to not be easily crushed. In an alternative configuration, the anti-suction component can be a series of interconnected balls, diamonds, beads or other interconnected objects or a chain or chain-like object that define the air flow channel around such objects.

The glove has a securing mechanism at the hand opening that is configured to secure the glove to the wearer's hand in a manner which prevents the glove from being removed by the wearer. In one embodiment, the securing mechanism comprises a pair of non-elastic straps and an attachment mechanism which attaches or otherwise joins the straps together. In another embodiment, one strap encircles the glove near the hand opening of the glove body and the attachment mechanism joins the ends of the strap together. The attachment mechanism can be a hook on one strap or strap end that is configured to engage a mesh panel on the other strap or strap end. In the preferred embodiment, the attachment mechanism is a side release buckle that comprises a first buckle which lockedly joins to a second buckle to secure the glove around the wrist area of the wearer's hand. The preferred embodiment of the glove also includes a mechanism cover that at least partially or fully covers the attachment mechanism in a manner which makes it much more difficult for the wearer to disengage the attachment mechanism by limiting the wearer's ability to use his or her sight or touch senses to figure out how to open, unlock, disconnect or otherwise disengage the attachment mechanism.

Accordingly, the primary aspect of the present invention is to provide a device for preventing sucking of the thumb or fingers that has the advantages discussed above and overcomes the disadvantages and limitations associated with the prior art anti-thumb or finger sucking devices.

It is also an important aspect of the present invention to provide a glove for preventing sucking of the thumb or fingers that is configured to prevent the wearer from obtaining a vacuum around the thumb or finger he or she likes to suck so as to discourage the wearer from sucking his or her thumb in order to prevent or break a sucking habit.

It is also an important aspect of the present invention to provide a glove for preventing sucking of the thumb or fingers having one or more tubular portions that extend substantially the entire length of a thumb or finger and one or more tubes that extend substantially the length of the tubular member to prevent the wearer from obtaining a vacuum around the thumb or finger.

It is also an important aspect of the present invention to provide a glove for preventing sucking of the thumb or fingers having a securing mechanism at the wrist portion of the glove that is configured to prevent the wearer from removing the glove and, thereby, defeating the anti-sucking benefits of the glove.

It is also an important aspect of the present invention to provide a glove for preventing sucking of the thumb or fingers that is configured to allow the wearer to utilize his or her hand and perform tasks therewith so as to not interfere with the wearer's dexterity development.

Another important aspect of the present invention is to provide a glove for preventing sucking of the thumb or fingers that is adaptable to being configured to prevent sucking of the thumb and/or one or more fingers.

Yet another important aspect of the present invention is to provide a glove for preventing sucking of the thumb or fingers that can be manufactured in a variety of different sizes and shapes to conform to the wearer's hand so that it may be utilized with different ages and sizes of wearers.

The above and other aspects and advantages of the present invention are explained in greater detail by reference to the attached figures and the description of the preferred embodiment which follows. As set forth herein, the present invention resides in the novel features of form, construction, mode of operation and combination of the above presently described and understood by the claims.

### BRIEF DESCRIPTION OF THE DRAWINGS

In the drawings which illustrate the preferred embodiments and the best modes presently contemplated for carrying out the present invention:

FIG. 1 is a top perspective view of a glove for preventing sucking of the thumb or fingers that is configured according to a preferred embodiment of the present invention for use to prevent sucking of the thumb;

FIG. 2 is a bottom perspective view of the glove of FIG. 1;

FIG. 3 is a top view of a glove for preventing sucking of the thumb or fingers that is configured according to a preferred embodiment of the present invention for use to prevent sucking of the first two fingers and shown on a wearer's left hand;

FIG. 4 is a bottom view of the glove of FIG. 3;

FIG. 5 is a bottom view of the glove of FIG. 4 shown with a plurality of gripping features thereon;

FIG. 6 is a top view of a glove for preventing sucking of the thumb or fingers that is configured according to a preferred embodiment of the present invention for use to prevent sucking of the second and third fingers and shown on a wearer's left hand;

FIG. 7 is a bottom view of the glove of FIG. 6;

FIG. 8 is a top view of a glove for preventing sucking of the thumb or fingers that is configured according to a preferred embodiment of the present invention for use to prevent sucking of the third and fourth fingers and shown on a wearer's left hand;

FIG. 9 is a bottom view of the glove of FIG. 8;

FIG. 10 is a side view of the preferred anti-suction component utilized with the glove of the present invention that is configured as a tube;

FIG. 11 is a side view of an alternative embodiment of the anti-suction component configured as a tube with a plurality of apertures;

FIG. 12 is a side view of another alternative embodiment of the anti-suction component configured as a plurality of interconnecting balls shown disposed in an affected member on an affected digit;

FIG. 13 is a side view of another alternative embodiment of the anti-suction component configured as a plurality of interconnecting half balls;

FIG. 14 is a side view of another alternative embodiment of the anti-suction component configured as a plurality of interconnecting half diamonds;

FIG. 15 is a side view of another alternative embodiment of the anti-suction component configured as a chain shown disposed in an affected member on an affected digit;

FIG. 16 is a side view of another alternative embodiment of the anti-suction component configured as a plurality of interconnected beads shown disposed in an affected member on an affected digit;

FIG. 17 is a top view of a connecting mechanism utilized to secure the glove of the present invention around the wrist of the user;

FIG. 18 is a top view of a glove for preventing the sucking of the thumb and/or fingers having a securing mechanism comprising a side release buckle as the attachment mechanism with the attachment mechanism shown in its open position;

FIG. 19 is a top view of the glove of FIG. 18 showing the attachment mechanism in its closed position; and

FIG. 20 is a top view of the glove of FIG. 19 showing use of a mechanism cover over the closed attachment mechanism.

### DETAILED DESCRIPTION OF THE PREFERRED EMBODIMENTS

With reference to the figures where like elements have been given like numerical designations to facilitate the reader's understanding of the present invention, the preferred embodiments of the present invention are set forth below. The enclosed figures and drawings are merely illustrative of one or more of the preferred embodiments and, as such, represent one or more ways of configuring the present invention. Although specific components, materials, configurations and uses are illustrated, it should be understood that a number of variations to the components and to the configuration of those components described herein and in the accompanying figures can be made without changing the scope and function of the invention set forth herein. For instance, although the figures and description provided herein are directed to certain shapes and designs for the glove, those skilled in the art will readily understand that this is merely for purposes of simplifying the present disclosure and that the present invention is not so limited.

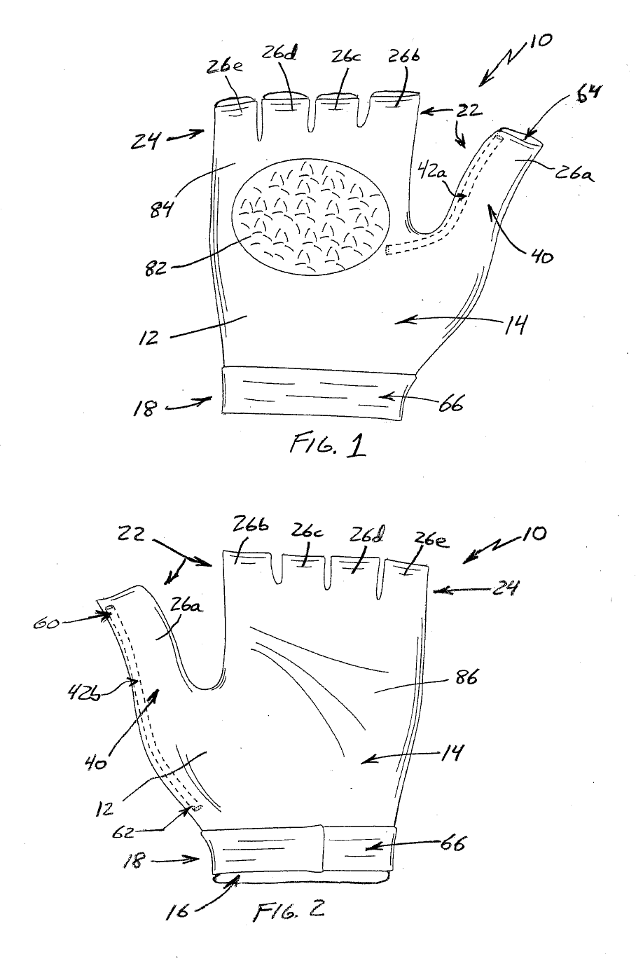

A glove for preventing sucking of the thumb and fingers that is manufactured out of the various components and configured pursuant to the preferred embodiments of the present invention is shown generally as 10 in FIGS. 1 through 9 and 18 through 20. As best shown in these figures, glove 10 generally comprises a glove body 12 that, as in a typical glove configuration, has a hand portion 14 with a hand opening 16 at the first end 18 of the glove body 12 for receiving the wearer's hand 20 and a digit portion 22 at the second end 24 of glove body 12 having a plurality of open ended digit receiving members 26, disposed generally opposite hand opening 16, that are each sized and configured to receive a digit therein, namely either thumb 28 or one of the first or pointer finger 30, second or middle finger 32, third or ring finger 34 and fourth or pinky finger 36. In the present disclosure, the term “digit” is utilized to refer to either the thumb 28 or any one of the fingers 30, 32, 34 and 36 and the term “digits” is utilized to refer to any combination of two or more of these digits. The digit portion 22 of glove body 12 comprises a thumb receiving member 26a for thumb 28, a first finger receiving member 26b for first finger 30, a second finger receiving member 26c for second finger 32, a third finger receiving member 26d for third finger 34 and a fourth finger receiving member 26e for fourth finger 36. As best shown in FIG. 1, the distal end of each of digit receiving members 26 is open to allow the end of the respective digit to extend past the end of their respective digit receiving member 26 to facilitate the wearer being able to fully utilize his or her hand 20 for eating, playing, drawing, writing and the like so that he or she will be able to fully develop their motor and dexterity skills.

As will be readily understood by those skilled in the art, glove body 12 is configured to generally conform to the shape of a typical hand 20 and to be generally worn in much the same way as a conventional, albeit fingerless, glove. Although glove body 12 can be made out of a wide variety of different materials, any material selected for glove body 12 must be substantially breathable, or at least not completely closed to air passing through the material, to prevent the wearer from achieving a vacuum against the outer surface of glove 10. Being breathable also helps avoid skin problems that could otherwise arise as a result of wearing a fully closed cover for an extended period of time. In addition, the preferred material for glove body 12 is one that is substantially flexible so glove 10 will not impede the wearer's motor skill usage and dexterity development, comfortable fitting so glove 10 can be worn for extended periods of time without causing discomfort for the wearer and relatively soft so glove 10 will not chafe or otherwise irritate the wearer. An example of such a material is cloth fabric.

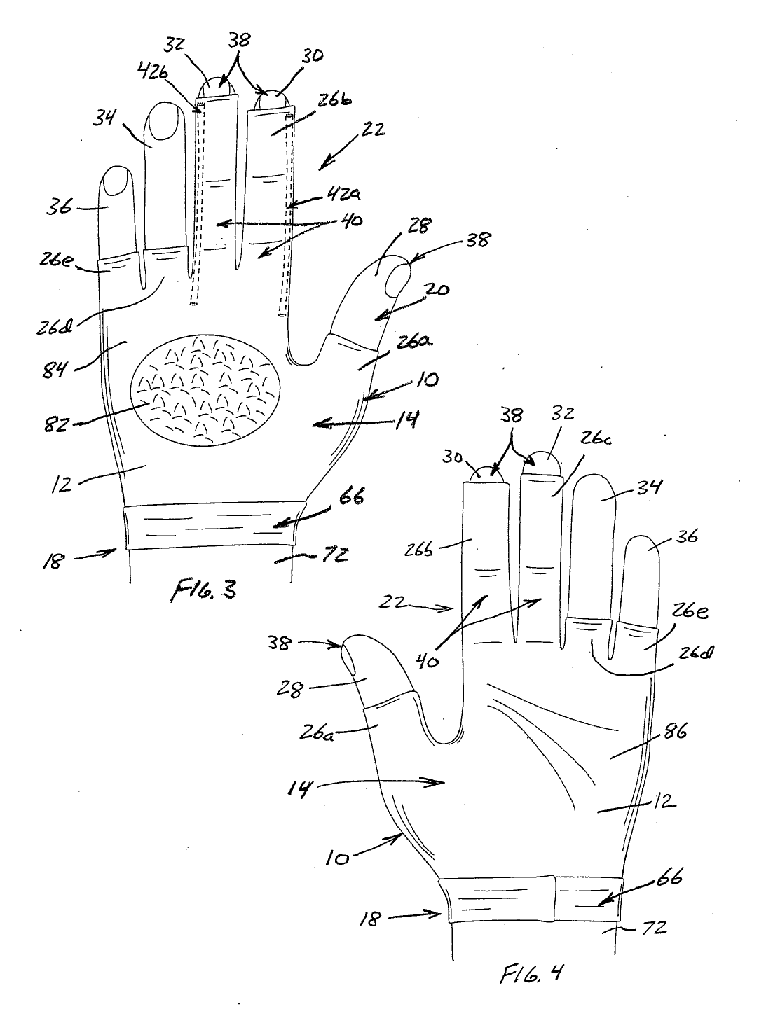

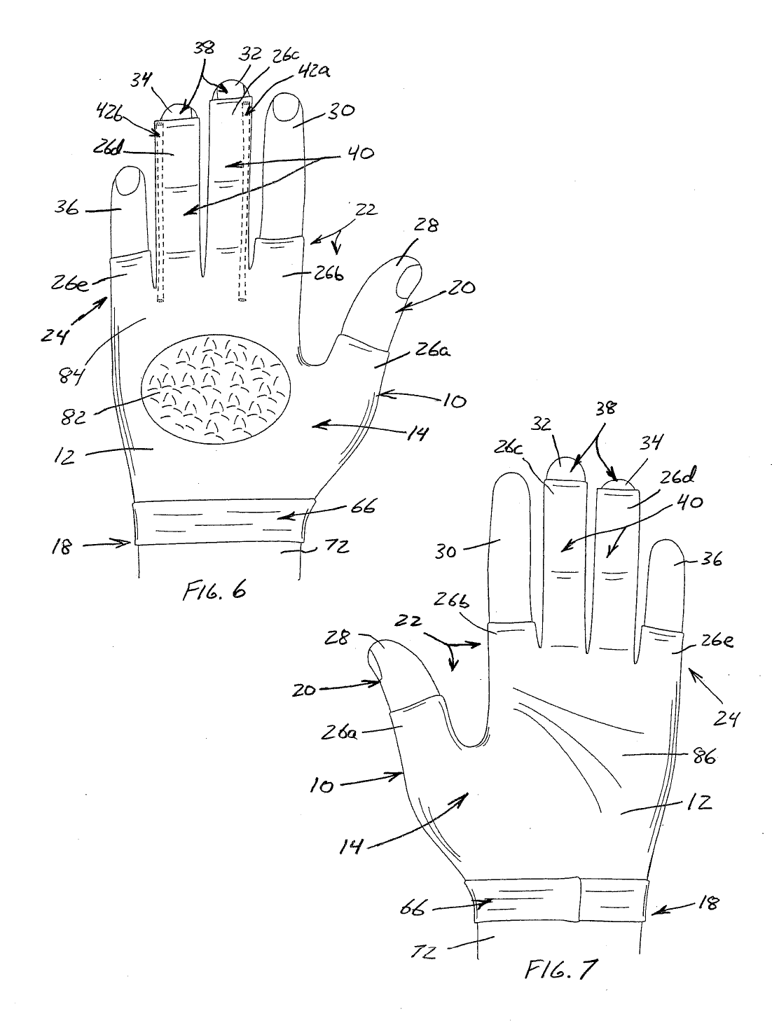

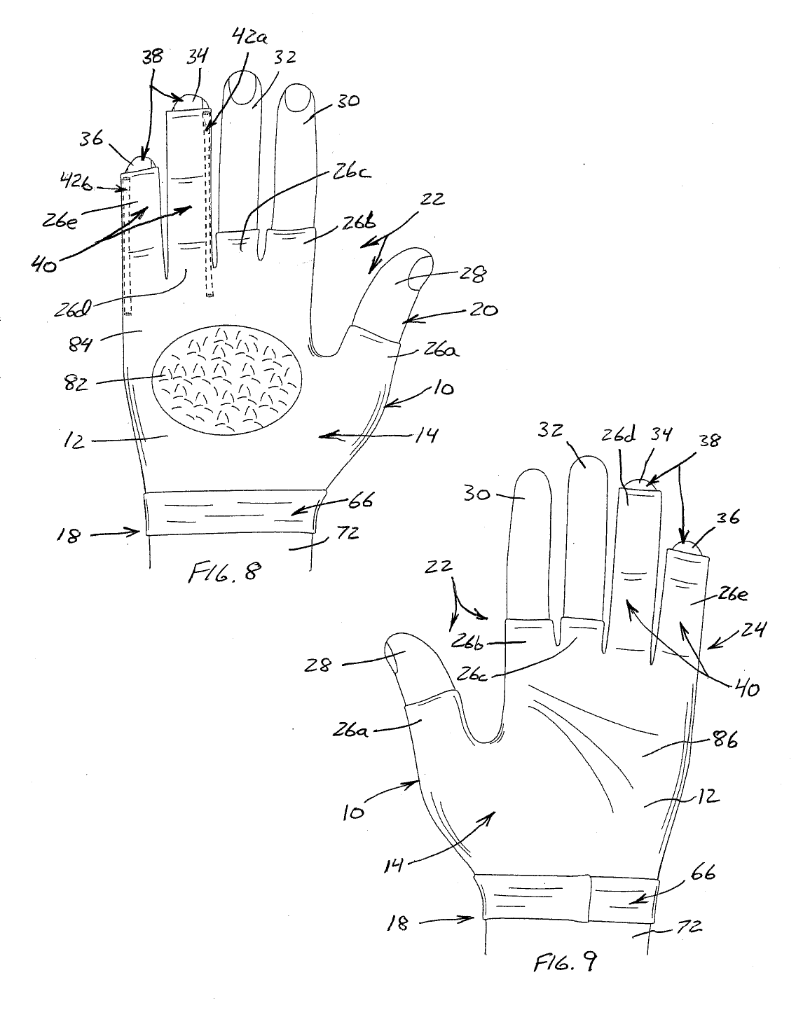

As shown in FIGS. 1 through 9, typically a number of the digit receiving members 26 only extend to or slightly beyond the base of their respective digit. For instance, in the embodiment shown in FIGS. 1 and 2 the receiving members 26b, 26c, 26d and 26e are configured to only extend slightly past the base of first finger 30, second finger 32, third finger 34 and fourth finger 36, respectively, whereas the receiving member 26a is configured to extend substantially the full length of its respective digit, namely thumb 28. In this embodiment, thumb 28 (not shown in these figures) is identified as the affected digit 38, as set forth below, and its corresponding receiving member 26a is designated as the affected member 40. As well known by those familiar with persons who suck their thumb 28 or fingers 30-36, a thumb/finger sucking person will primarily suck either their thumb 28 or one or more of their fingers 30-36, typically sucking two or more of the fingers 30-36. Most thumb/finger sucking individuals develop a sucking routine such that he or she does not usually switch between thumb 28 and one or more of the fingers 30-36. As such, it is generally only necessary to configure glove 10 with sufficient affected members 40, which extend substantially the full length of the affected digit(s) to prevent suction of the affected digit(s) 38. As stated above, in the embodiment of FIGS. 1 and 2, which are configured for a thumb sucker, the affected digit 38 is thumb 28 (not shown in these figures) and the affected member 40 is digit receiving member 26a. For persons who suck the first two fingers, first finger 30 and second finger 32, of their hand 20, the glove 10 is configured as shown in FIGS. 3, 4 and 5, with first 30 and second 32 fingers being the affected digits 38 and the digit receiving members 26b and 26c being the affected members 40. For persons who suck their second two fingers, second finger 32 and third finger 34, of their hand 20, the glove 10 is configured as shown in FIGS. 6 and 7, with second 32 and third 34 fingers being the affected digits 38 and digit receiving members 26c and 26d being the affected members 40. For persons who suck their third two fingers, third finger 34 and fourth finger 36, of their hand 20, the glove 10 is configured as shown in FIGS. 8 and 9, with third 34 and fourth 36 fingers being the affected digits 38 and digit receiving members 26d and 26e being the affected members 40. Although not shown, the glove 10 can also be configured for those persons who suck three fingers, such as first 30, second 32 and third 34 fingers or second 32, third 34 and fourth 36 fingers (as the affected digits 38), by utilizing digit receiving members 26b, 26c and 26d or digit receiving members 26c, 26d and 26e, respectively, as the affected members 40. As shown in FIGS. 1 through 9, the digit receiving members 26 that are not the affected members 40 do not extend the full length of their respective digits so as to allow the wearer of glove 10 as much finger freedom as possible while still preventing him or her from sucking their affected digits 38. If desired, glove 10 can be configured such that each of digit receiving members 26a-26e are configured as affected members 40 to prevent the wearer from sucking on any of the thumb 28 and fingers 30-36, as the affected digits 38.

In order to prevent the wearer of glove 10 from being able to obtain sufficient vacuum to suck their affected digits 38, each of the affected members 40 are provided with one or more anti-suction components 42 that, individually, define an air flow channel 44 inside the affected member 40 such that any attempt to suck on the affected digits 38 will be futile, effectively discouraging sucking activity so as to prevent or break a habit of such sucking. In a preferred embodiment, as set forth in more detail below, a single affected member has one or more anti-suction components, such as 42a and 42b of FIGS. 1 and 2, and each group or set of affected members 40 (i.e., two or more) will have at least one anti-suction component 42a or 42b for each of the affected members 40. Preferably, when a single affected member 40 is utilized, such as for the thumb 28 of FIGS. 1 and 2, the anti-suction components 42a and 42b are positioned on different sides of the affected member 40 (26a) so the wearer of glove 10 cannot pinch both of the anti-suction components 42a/42b closed at the same time by biting down on components 42a/42b at the same time. By positioning anti-suction components 42a/42b in affected member 40 such that they are along the sides of the affected digit (thumb 28), the typical thumb-sucking action does not lend itself to pinching the anti-suction components 42a/42b closed. In the embodiments where there are two or more affected members 40, such as those shown in FIGS. 3 through 9, each of the affected members 40 have at least one anti-suction component 42 and they are positioned such that the wear cannot pinch them both closed at the same time, such as by positioning an anti-suction component 42 on the outer sides (as shown) of the affected members 40 and, therefore, the outer sides of the affected digits 38. Any attempt to suck on the affected digit(s) 38, will result in the wearer merely sucking air through the air flow channel 44 created by the anti-suction component 42 and, as such, prevent him or her from being able to create a vacuum at the affected digit(s) 38. Over time, the wearer will be discouraged with such attempts and lose interest in sucking the affected digit(s) 38.

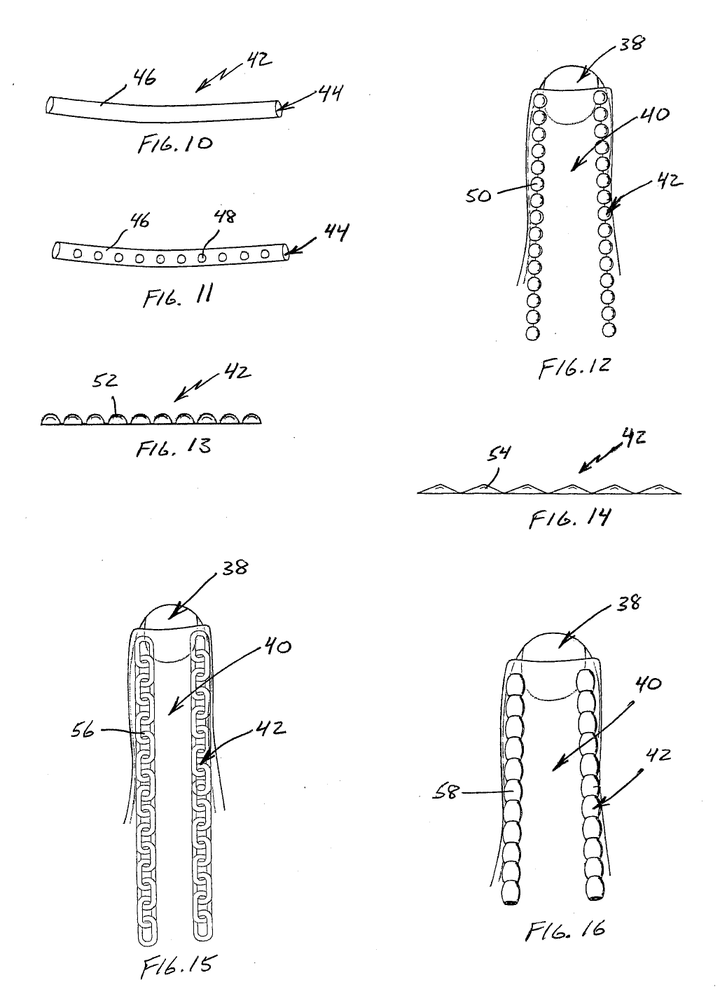

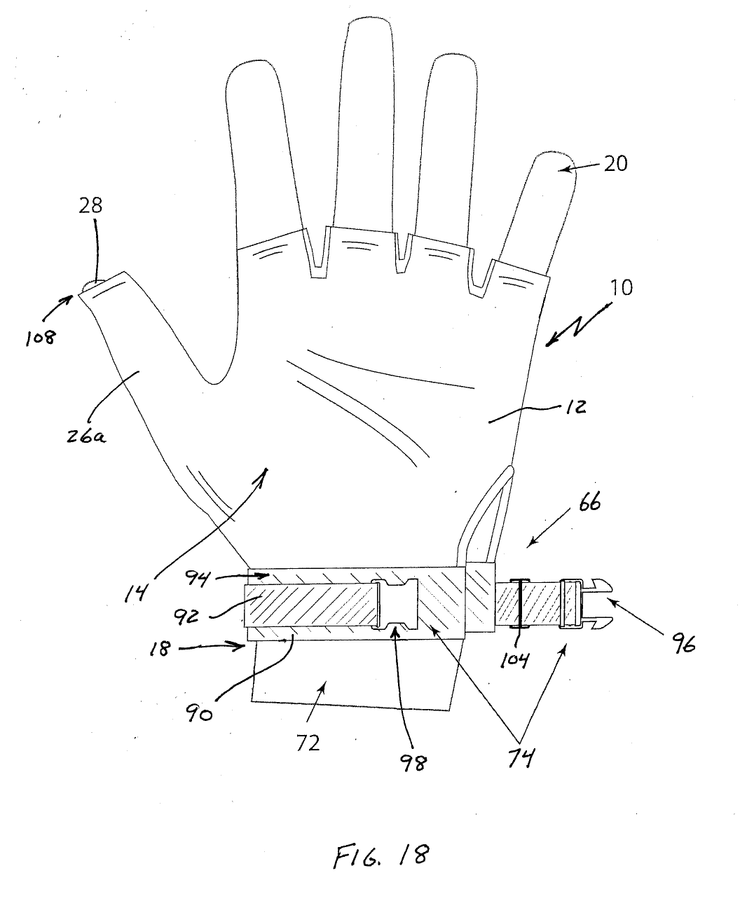

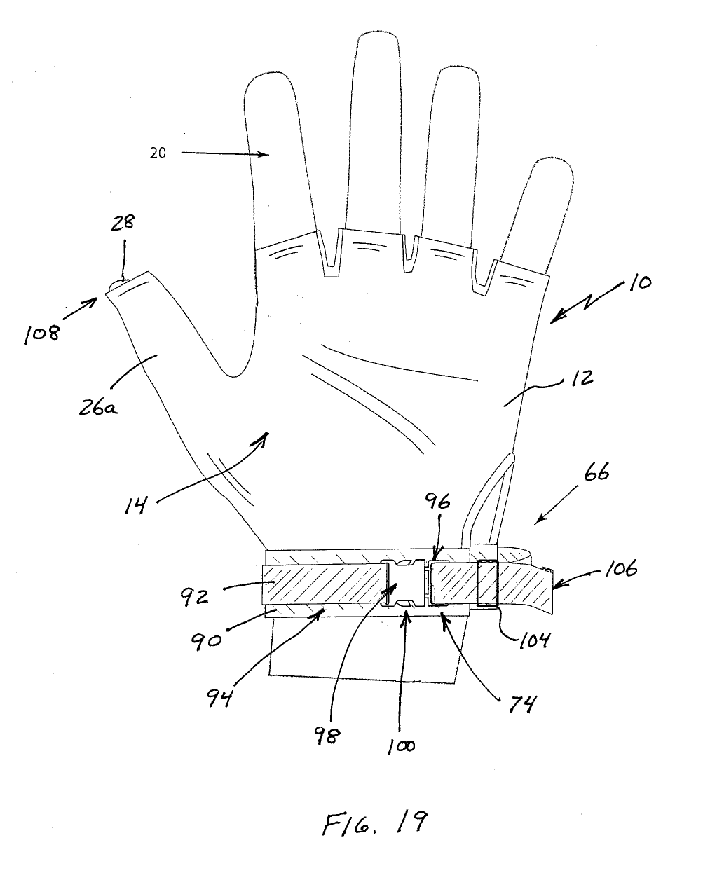

As shown in the embodiments of FIGS. 1 through 9, the preferred anti-suction component 42 is a tube 46, which is best shown in FIG. 10, with the air flow channel 44 being the interior of tube 46. The tube 46 should be made of a material, such as plastic or the like, which is sufficiently stiff such that tube 46 is not easily pinched or crushed with the wearers teeth or his or her non-sucking hand, which would close the air flow channel 44 therein and allow the wearer to create a vacuum at the affected digit(s) 38, thereby defeating the various sucking prevention aspects of glove 10 of the present invention. As will be readily appreciated by those skilled in the art, tube 46 is likely the preferred anti-suction component 42 due to its well defined and open air flow channel 44. FIG. 11 shows an alternative embodiment of tube 46 where the tube 46 has a plurality of apertures 48 along the sides thereof to even more fully allow air into the air flow channel 44 of tube 46 to prevent the wearer from forming a vacuum along affected member 40. Various other alternative embodiments for anti-suction component 42 are shown in FIGS. 12 through 16. In FIG. 12, the anti-suction component 42 is a series of interconnected small balls 50 that form an air flow channel 44 around the sides of the balls 50 when disposed inside the affected member 40. FIG. 13 shows the use of semi-spherical balls 52 that can be utilized as the anti-suction component 42, typically with the flat sides thereof positioned so as to be against the affected digit 38. FIG. 14 shows the use of a half diamonds 54 as the anti-suction component 42. As with the semi-spherical balls 52, the flat sides of the half diamonds 54 can be placed against the affected digit and the triangle-shaped side can form the air flow channel 44 through the affected member 40. In FIG. 15, the anti-suction component 42 is a chain 56 having interconnected chain links that form an air flow channel 44 around the sides of the chain 56 when disposed inside the affected member 40. FIG. 16 shows use of a plurality of interconnected beads 58, typically in the form of a string of beads 58, as the anti-suction component 42 to form air flow channel 44 around the sides of the beads 58 when disposed inside affected member 40.

As stated above, each of the affected members 40 will have at least one anti-suction component 42 disposed along a portion of the length of affected member 40. In a preferred embodiment, the anti-suction component 42 extends substantially the entire length of the affected member 40, from near its distal end, where affected digit 38 extends outwardly therefrom, to the hand portion 14 of glove body 12, as exemplified in FIGS. 1 and 2. In this manner, when the wearer attempts to such his or her affected digit 38, air will flow along the air flow channel 44 inside the anti-suction component 42, such as tube 46, from the hand portion 14 of glove body 12. The length of anti-suction component 42 prevents the wearer from extending his or her mouth over both of the open end of the tube 46 at or near the hand portion 14 of glove body 12, which would allow the wearer to obtain sufficient vacuum to suck his or her affected digit 38. For purposes of the present disclosure, the end of the anti-suction component 42 at the distal end of the affected member 40 is referred to as the first end 60 and the end of the anti-suction component 42 extending toward or into hand portion 14 is referred to as the second end 62. In a preferred embodiment, as shown in FIGS. 1 through 9, first end 60 of anti-suction component 42 terminates before reaching the open end 64 of the affected member 40 and the second end 62 of anti-suction component 42 terminates in the hand portion 14 of glove body 12. Positioning the first end 60 of the anti-suction component 42 such that it terminates prior to the open end 64 of affected member 40 is preferred because the material of the affected member 40 over the open first end 60 prevents anti-suction component 42 from becoming clogged and, therefore, unable to pass air through the air flow channel 44. As stated above, positioning the open second end 62 of anti-suction component 42 in the hand portion 14 prevents the wearer from simultaneously closing both ends 60/62 at the same time with his or her mouth. If desired, however, the length of the anti-suction component 42 can be selected such that the second end 62 thereof is disposed in the affected member 42, but of a sufficient length to prevent the wearer from closing both ends 60/62 with his or her mouth.

To secure the glove 10 onto the wearer's hand 20 so that he or she will not be able to easily remove it from the hand 20 and, therefore, make his or her affected digits 38 available for sucking, the glove 10 of the present invention is provided with securing mechanism 66. The securing mechanism 66 should be of the type that a parent or other responsible person can utilize to secure glove 10 to the hand 20 but that the wearer cannot easily manipulate to remove. In a preferred embodiment, the securing mechanism 66 comprises a pair of strap members, first strap member 68 and second strap member 70 (which can be separate strap members or the opposite ends of a single strap), that are sized to join around the wrist area 72 of wearer's hand 20 and an engaging mechanism 74 that engagedly connects the two strap members 68/70 together, as shown in FIGS. 17 through 20, In the embodiment shown in FIG. 17, engaging mechanism 74 comprises a hook 76 on the underside of the first strap member 68 and a hook-receiving mesh 78, which can be configured as a series of mesh panels or loops, on the top side of second strap member 70 that are configured such that the hook 76 securely engages the hook-receiving mesh 78 when engaging mechanism 74 is engaged. The hook 76 and mesh 78 should be chosen such that it is relatively difficult for most wearers of glove 10 to remove glove 10 on their own. The straps 68/70 should be of the type that are non-elastic to prevent glove 10 from being pulled off of the hand 20 of the wearer. If desired, securing mechanism 66 can include a strap flap or handle 80 to assist the parent or like with the installation and removal of glove 10 from the wearer's hand 20.

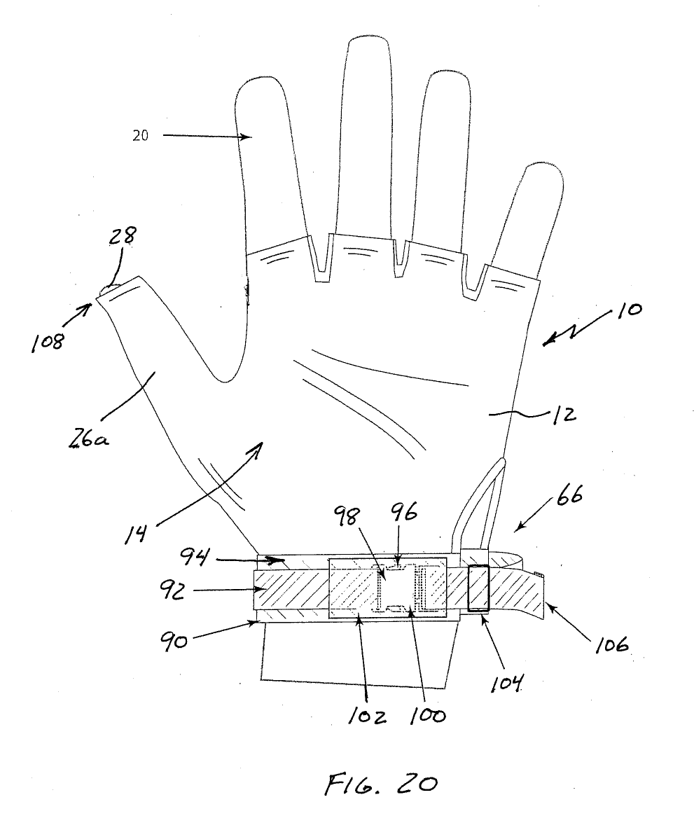

In a preferred embodiment, the securing mechanism 66 comprises a comfort strap 90 that is disposed around or at least substantially around the first end 18 of the glove body 12 of glove 10 and a buckle strap 92 that is positioned on the outer surface 94 of comfort strap 90, as shown in FIGS. 18 through 20, and attached or secured thereto by stitching, belt loops or other mechanisms. Both the comfort strap 90 and buckle strap 92 are sized and configured to go around the wrist area 72 of the wearer's hand 20. The engaging mechanism 74 of this embodiment of securing mechanism 66 is configured to engagedly connect the two ends of buckle strap 92 together. In the embodiment of FIGS. 18 through 20, the engaging mechanism 74 comprises a first or male clip member 96 and a second or female clip member 98, best shown in FIG. 18, that are cooperatively configured to engagedly connect to each other, as shown in FIGS. 19 and 20. In this embodiment, the engaging mechanism 74 (which is commonly known as a side release buckle) is configured such that the first clip member 96 snaps into the second clip member 98 to lockedly engage the engaging mechanism 74 in its closed position 100 to substantially close the first end 18 of glove body 12 around the wrist area 72 of the wearer's hand 20. Depending on the configuration and manufacture of glove 10, the engaging mechanism 74 of this embodiment may be made in a manner that requires a high amount of pressure to release or with an internal or external locking mechanism that is difficult enough to unfasten so as to prevent glove 10 from removed from the wearer's hand 20, which by itself may be sufficient to secure glove 10 to hand 20.

To ensure securing mechanism 66 is effective and that the engaging mechanism 74, best shown in its closed position 100 in FIG. 19, is difficult if not nearly impossible to unfasten by anyone other than a parent or other responsible person, glove 10 can be provided with a mechanism cover 102 that is attached to either comfort strap 90 or glove 10 in a way which covers, or at least substantially covers, and hides the second side 98 of the engaging mechanism 74. With the mechanism cover 102 positioned over the engaging mechanism 74 (as shown in FIG. 20) when it is in its closed position 100, the wearer of glove 10 will not be able to see engaging mechanism 74, thereby making it difficult for the wearer to understand how the engaging mechanism 74 functions (e.g. how it is fastened together and unfastened), which will make it more difficult for the wearer to remove from his or her hand 20. As will be readily appreciated by those skilled in the art, the configuration of the engaging mechanism 74 of this embodiment could be reversed, such that the first/male clip member 96 could be substituted for the second/female clip member 98 and vice versa.

To further strengthen the effectiveness of the glove 10, mechanism cover 102 can be made of a material of having sufficient thickness and stiffness to prevent the wearer from discovering how engaging mechanism 74 works by using his or her touch sensation to feel the buckle or other engaging mechanism 74. One such material for use in mechanism cover 102 is nylon webbing, but it can be any material such as leather, vinyl or other synthetic or natural fabrics or other non-fabric materials that will prevent the wearer from using a great deal or all of the touch sense of his or her fingers to feel the shape of engaging mechanism 74 underneath mechanism cover 102 and thus figure out how to open or otherwise disengage the engaging mechanism 74. Additionally, the mechanism cover 102 is to be made of a sufficiently thick material as to make it difficult for the wearer to unfasten the clip members 96/98 or other components of engaging mechanism 74 in order to remove the glove 10. As will be appreciated by those skilled in the art, the mechanism cover 102 will require extra pressure, which will not be able to be supplied by the wearer, needed on buckle type of engaging mechanism 74 to squeeze in the sides and release engaging mechanism 74, as would be done in typical use of such mechanisms. Buckle strap 92 should be made of a material that is non-elastic to prevent the glove 10 from being pulled off of the hand 20 of the wearer without properly releasing engaging mechanism 74. The sizing of buckle strap 92 can be adjusted by the use of a ladder lock 104, shown in FIGS. 18 through 20.

If desired, the engaging mechanism 74 shown in the figures can be replaced with other designs of buckles or fasteners or locking buckles or locking fasteners that effectively prevent the removal of glove 10 from the wearer's hand 20 and which, in order to be effective, may or may not require objects similar or identical to mechanism cover 102. If desired, the two-strap design that features buckle strap 92 and comfort strap 94 can be combined into a single strap design. This design of securing mechanism 66 is applicable to a wide variety of designs of glove 10 that may or may not include covers for any and all digits of wearer's hand 20 in all possible combinations or permutations of digit coverage. As an added functional and safety feature, the ladder lock 104 can be secured onto buckle strap 92 by folding over and stitching the end 106 of buckle strap 92 a sufficient number of times to build up enough thickness to prevent buckle strap 92 from slipping out of ladder lock 104, which would allow the glove 10 to be removed from the wearer's hand 20 to due the slack in buckle strap 92 that would result or due to the removal of ladder lock 104 from buckle strap 92, causing the securing mechanism 66 to become ineffective. As will be appreciated by those skilled in the art, other configurations of securing mechanism 66 and engaging mechanism 74, some of which are described in the above-identified prior art or otherwise known to those skilled in the art, can also be utilized with glove 10 of the present invention.

Uses of the securing mechanism 66 shown in FIGS. 18 through 20 are not limited to only the glove 10 of the present invention. Securing mechanism 66, with specific consideration to the use of a side release buckle as the engaging mechanism 74 in conjunction with a concealing cover such as mechanism cover 102 to make it more difficult for persons to disengage or otherwise remove the engaging mechanism 74 due to the visual disguising, touch sensation disguising and added pressure of removal, can be applied to other devices such as but not limited to backpacks, dog collars, medical devices, shoes, child car seats, strollers or any other products where a difficult to remove fastening system is required.

If desired, one or more decorative items 82 can be placed on the upper surface 84 of glove body 12, as shown in FIGS. 1, 3, 6 and 8 or, though not preferred due to possible interference with the use of hand 20, on the palm surface 86 of glove body 12. In the preferred embodiment, the decorative items 82 are removably attached to glove body 12 so that the parent or wearer can change them out as desired for a different look. Decorative item 82 can be attached utilizing Velcro®, snaps, buttons or other connectors that are appropriate for the age and mentality of the wearer of glove 10. Typically, the decorative item will be of the type that is appealing to children.

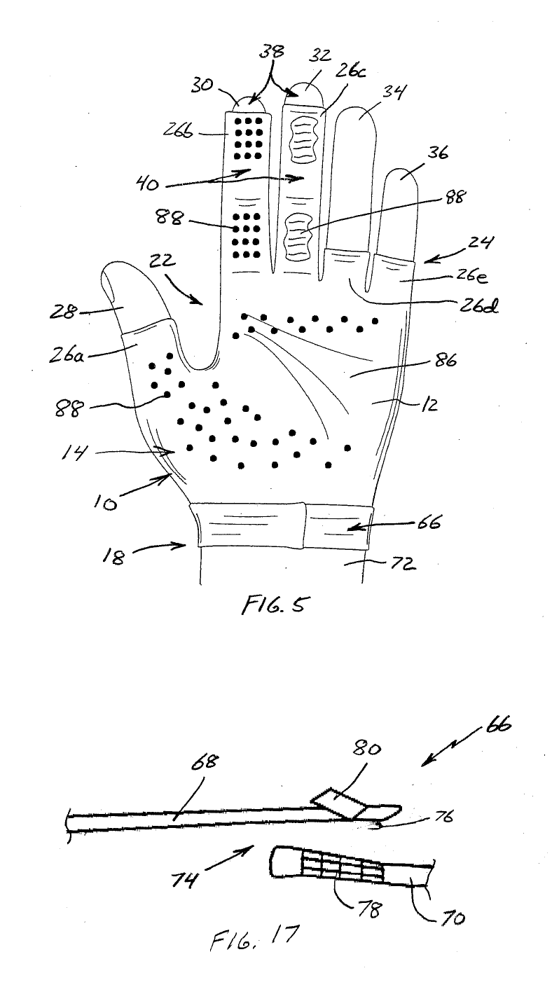

If also desired, glove 10 can include one or more gripping members 88 to improve the gripping ability of the glove 10, as shown in FIG. 5. Typically, the gripping members 88 will be placed on the palm side surface 86 of glove body 12 (as shown) and may be positioned on the hand portion 20 and/or the digit portion 22, including the affected members 40. As familiar to those skilled in the art, a wide variety of gripping members 88 can be utilized with glove 10 to at least generally improve the ability of a person wearing glove 10 to grip an object in his or her hand. Examples of some of the gripping members 88 that can be utilized with glove 10 and patterns of those gripping members 88 on the palm surface 86 of glove body 12 are shown in FIG. 5. A variety of other gripping members 88 can also be utilized with the glove 10 of the present invention.

In use, glove 10 is manufactured with a glove body 12 configured to generally conform to the intended wearer's hand 20 with the desired configuration of affected members 40 to prevent the wearer from sucking on the affected digits 38. For some wearer's this will be a thumb 28 configuration, such as shown in FIGS. 1 and 2, and for other wearers this will be a combination of various fingers 30-36, such as shown in FIGS. 3 through 9. Typically, the non-affected digits will extend beyond the open end of digit receiving members 26. For some wearers, namely those who suck on their thumb 28 and fingers 30-36, glove 10 will have affected members 40 in place of all of the digit receiving members 26. In the embodiment of FIGS. 18 through 20, the thumb digit receiving member 26a extends nearly all of the way to the distal end 108 of thumb 28 to even more effectively prevent sucking of the thumb 28. A parent or other responsible person places the wearer's hand 20 into glove body 12 though the hand opening 16 and his or her fingers through the digit receiving members 26 and affected members 40 and then utilizes the securing mechanism 66 to secure the glove 10 to the wearer's hand 20 in a manner that the wearer himself or herself cannot easily undo. If applicable, mechanism cover 102 is then positioned over the engaging mechanism 74 to hide or at least substantially hide the engaging mechanism 74. Once in place, the wearer will substantially utilize his or her hand 20 as they normally would, except that if he or she attempts to suck on his or her favorite thumb 28 and/or fingers 30-36 they will not be able to obtain a vacuum against the affected member 40 and, therefore, will not be able to suck the intended affected digit 38. Instead, air will be drawn through the air flow channel 44 of the anti-suction component 42 in a manner that is similar to sucking on a straw, thereby preventing the vacuum necessary to suck. As a result the wearer of glove 10 will quickly become very discouraged with his or her sucking attempts. Before long, use of the glove 10 will break a habit of sucking a thumb 28 or fingers 30-36 or prevent formation of such a habit.

While there are shown and described herein specific forms of the invention, it will be readily apparent to those skilled in the art that the invention is not so limited, but is susceptible to various modifications and rearrangements in design and materials without departing from the spirit and scope of the invention. In particular, it should be noted that the present invention is subject to various modification with regard to any dimensional relationships set forth herein and modifications in assembly, materials, size, shape and use. For instance, there are numerous components described herein that can be replaced with equivalent functioning components to accomplish the objectives of the present invention.

What is claimed is:

1. A glove for preventing sucking of one or more affected digits of a hand, said glove comprising:
a glove body configured to generally conform to the shape of the hand, said glove body having a hand portion with a hand opening at a first end of said glove body for receiving the hand therein and a digit portion with a plurality of tubular open ended digit receiving members at a second end of said glove body disposed generally opposite said hand opening for receiving a digit of the hand therein, each of said digit receiving members corresponding to the affected digits being designated an affected member and configured to extend substantially the full length of its corresponding affected digit; and
one or more anti-suction components along a length of each of said affected members, each of said anti-suction components configured to provide an air flow channel through said affected member.

2. The glove according to claim 1, wherein said glove body is made from a breathable material.

3. The glove according to claim 1, wherein said affected member is made from breathable material and each of said anti-suction components are embedded in said breathable material.

4. The glove according to claim 1, wherein said affected digit is a thumb and said affected member is configured to extend substantially the full length of said thumb, said affected member configured with a pair of anti-suction components disposed in spaced apart relation to each other.

5. The glove according to claim 4, wherein each of said pair of anti-suction components have a first end that is disposed toward an open end of said affected member and a second end that is disposed toward or in said hand portion of said glove body, said first end of said anti-suction component not extending fully to said open end of said affected member.

6. The glove according to claim 1, wherein said air flow channel extends into said hand portion of said glove body and is configured to draw air from said hand portion.

7. The glove according to claim 1, wherein said anti-suction component is a tube and said air flow channel is disposed inside said tube.

8. The glove according to claim 1, wherein said anti-suction components extend substantially the full length of said affected member.

9. The glove according to claim 8, wherein said anti-suction component has a first end that does not extend to an open end of the affected member and is completely enclosed by a breathable material.

10. The glove according to claim 1, wherein said affected member comprises a pair of anti-suction components in spaced apart relation to each other.

11. The glove according to claim 1 further comprising means at said first end of said glove body for removably securing said glove body to the hand, said securing means configured to prevent easy removal of said glove body from the hand.

12. The glove according to claim 11, wherein said securing means comprises one or more non-elastic strap members and an engaging mechanism associated with at least one of said strap members, said engaging mechanism configured to substantially close said hand opening of said glove body around a wrist area of the hand.

13. The glove according to claim 12, wherein said engaging mechanism comprises a hook and a mesh panel, said hook configured to be engaged with said mesh panel.

14. The glove according to claim 12, wherein said engaging mechanism comprises a comfort strap and a buckle strap disposed over an outer surface of said comfort strap, said buckle strap having a first clip member that is configured to lockingly engage a second clip member.

15. The glove according to claim 12 further comprising a mechanism cover configured to at least substantially cover said engaging mechanism when it is in a closed position so as to increase the difficulty of disengaging said engaging mechanism.

16. A glove for preventing sucking of one or more affected digits of a hand, said glove comprising:
a glove body configured to generally conform to the shape of the hand, said glove body made out of a breathable material and having a hand portion with a hand opening at a first end of said glove body for receiving the hand therein and a digit portion with a plurality of tubular open ended digit receiving members at a second end of said glove body disposed generally opposite said hand opening for receiving a digit of the hand therein, each of said digit receiving members corresponding to the affected digits being designated an affected member and configured to extend substantially the full length of its corresponding affected digit;
one or more anti-suction components associated with each affected member, said anti-suction components disposed substantially along the entire length of said affected member but not to the open end of said affected member, each of said anti-suction components configured to provide an air flow channel through said affected member; and
means at said first end of said glove body for removably securing said glove body to the hand, said securing means configured to prevent easy removal of said glove body from the hand.

17. The glove according to claim 16, wherein said air flow channel extends into said hand portion of said glove body and is configured to draw air from said hand portion.

18. The glove according to claim 16, wherein said anti-suction component is a tube and said air flow channel is disposed inside said tube.

19. The glove according to claim 16, wherein said securing means comprises one or more non-elastic strap members and an engaging mechanism associated with at least one of said strap members, said engaging mechanism configured to substantially close said hand opening of said glove body around a wrist area of the hand.

20. A glove for preventing sucking of one or more affected digits of a hand, said glove comprising:
a glove body configured to generally conform to the shape of the hand, said glove body made out of a breathable material and having a hand portion with a hand opening at a first end of said glove body for receiving the hand therein and a digit portion with a plurality of tubular open ended digit receiving members at a second end of said glove body disposed generally opposite said hand opening for receiving a digit of the hand therein, each of said digit receiving members corresponding to the affected digits being designated an affected member and configured to extend substantially the full length of its corresponding affected digit; and
one or more elongated tubes embedded in each affected member, each of said tubes disposed substantially along the entire length of said affected member but not to the open end of said affected member, each of said tubes having an air flow channel therein.
